(19) United States
(12) Patent Application Publication
(10) Pub. No.: US 2026/0315289 A1
(43) Pub. Date: Oct. 15, 2026

(54) URINARY INCONTINENCE PROTECTION PAD

(71) Applicant: Christopher Loren Graham, Fitchburg, MA (US)

(72) Inventor: Christopher Loren Graham, Fitchburg, MA (US)

(21) Appl. No.: 19/647,774

(22) Filed: Apr. 14, 2026

Related U.S. Application Data
(60) Provisional application No. 63/788,593, filed on Apr. 14, 2025.

(51) Int. Cl. A61F 13/471; A61F 13/15; A61F 13/56

(52) U.S. Cl. CPC A61F 13/471; A61F 13/5611; A61F 2013/15121; A61F 2013/15146

(57) ABSTRACT

A male urinary incontinence protection pad including an elongated tubular sleeve comprising absorbent material and having an opening at a first end, the elongated tubular sleeve being sized to receive at least a portion of a penis through the opening, and an elastic ring disposed around the opening and configured to elastically engage the penis to retain the elongated tubular sleeve on the penis and inhibit leakage of urine from the opening, wherein the elongated tubular sleeve is configured to absorb urine released from the penis.

### CROSS REFERENCE TO RELATED APPLICATIONS

This application claims the benefit of and priority to U.S. Provisional Patent Application No. 63/788,593, filed Apr. 14, 2025, and entitled “URINE COLLECTION PAD,” the entire disclosure of which is incorporated herein by reference to the extent permitted.

### BACKGROUND OF THE INVENTION

### 1. Field of the Invention

The present disclosure relates generally to absorbent personal care articles and urinary incontinence products. More particularly, the present disclosure relates to a wearable male urinary incontinence protection pad having an elongated tubular absorbent body configured to be worn on a penis to absorb urine and help protect clothing, bedding, and surrounding materials during nighttime use, travel, and other extended-use situations.

### 2. Description of Related Art

Male users may experience urinary incontinence, urinary leakage, bedwetting, accidental urine release, or reduced bladder control in a variety of situations. Such incidents may occur during nighttime sleep, while traveling, during extended trips, in hospital or care settings, or in other circumstances in which immediate restroom access is limited, delayed, or unavailable.

Urinary leakage or involuntary urination may soil clothing, bed linens, blankets, mattresses, seating surfaces, vehicle interiors, medical linens, or other surrounding materials. In addition to the inconvenience associated with cleaning or replacement, these incidents may cause embarrassment, anxiety, discomfort, and reduced willingness to participate in normal daily, occupational, social, overnight, recreational, or travel-related activities.

Some known incontinence or urine management products may be bulky, uncomfortable, difficult to attach, difficult to conceal, or not well suited for temporary, discreet, and wearable protection directly on the male anatomy. Some products may also be prone to leakage or may not provide a simple structure that can be applied quickly, worn discreetly, and removed after use.

Accordingly, there is a need for a simple, discreet, and effective male urinary incontinence protection pad that can be worn on the penis, absorb urine leakage or involuntary urination, help inhibit leakage from the device, and protect adjacent materials during use.

There is also a need for a male urinary incontinence protection pad that is suitable for nighttime use, bedwetting protection, long trips, extended travel, temporary wear, and other situations in which a user may desire confidence and protection without relying on a bulky or complicated product.

### SUMMARY OF THE DISCLOSURE

In one aspect, a male urinary incontinence protection pad comprises an elongated tubular sleeve comprising absorbent material and having an opening at a first end, the elongated tubular sleeve being sized to receive at least a portion of a penis through the opening, and an elastic ring disposed around the opening and configured to elastically engage the penis to retain the elongated tubular sleeve on the penis and inhibit leakage of urine from the opening, wherein the elongated tubular sleeve is configured to absorb urine released from the penis.

In some embodiments, the elongated tubular sleeve comprises an absorbent layer.

In some embodiments, the elongated tubular sleeve comprises an absorbent core.

In some embodiments, the elongated tubular sleeve further comprises an inner leak-resistant barrier and an outer leak-resistant barrier.

In some embodiments, the absorbent core is disposed between the inner leak-resistant barrier and the outer leak-resistant barrier.

In some embodiments, the absorbent material comprises one or more of an absorbent chemical, a gel, a superabsorbent polymer, or combinations thereof.

In some embodiments, the urinary incontinence protection pad further comprises an elastic band portion disposed on the elongated tubular sleeve and configured to further secure the elongated tubular sleeve on the penis.

In some embodiments, the urinary incontinence protection pad is configured for nighttime use, bedwetting protection, long trips, travel use, hospital use, or other extended-wear or temporary-wear situations.

In some embodiments, the urinary incontinence protection pad includes at least a portion that is coated with a glow in the dark coloring to be visible at night.

In some embodiments, the urinary incontinence protection pad includes at least a portion that is coated with a glow in the dark coloring to be visible at night, only when the absorbent material has been exposed to urine.

In some embodiments, the urinary incontinence protection pad is configured to help protect clothing, bedding, seating surfaces, linens, or other surrounding materials from urine leakage or involuntary urination.

In some embodiments, the urinary incontinence protection pad may be removed after use, rolled up, and discarded in a sanitary manner.

In another aspect, a urinary incontinence protection device for a male user comprises a sleeve body defining an elongated tubular sleeve having a proximal opening, an absorbent core carried by the sleeve body, and an elastic ring surrounding the proximal opening and configured to secure the sleeve body to a penis and reduce leakage at the proximal opening, wherein the sleeve body comprises an inner leak-resistant barrier and an outer leak-resistant barrier positioned relative to the absorbent core to retain urine absorbed by the absorbent core.

In another aspect, a method of providing urinary incontinence protection for a male user comprises placing a penis of the male user through an opening of a urinary incontinence protection device such that the penis is engaged by an elastic ring surrounding the opening, retaining the device on the penis with the elastic ring, and absorbing urine released from the penis within an elongated tubular sleeve of the device.

Additional aspects, embodiments, features, and advantages of the present disclosure will become apparent from the following description and the appended claims.

### BRIEF DESCRIPTIONS OF THE DRAWINGS

These and/or other aspects of the present general inventive concept will become apparent and more readily appreciated from the following description of the embodiments, taken in conjunction with the accompanying drawings of which:

FIG. 1 is a front perspective view a urinary incontinence protection pad according to an embodiment of the present inventive concept;

FIG. 2 is a top view the urinary incontinence protection pad illustrated in FIG. 1;

FIG. 3 is a bottom view of the urinary incontinence protection pad illustrated in FIG. 1;

FIG. 4 is a side view of the urinary incontinence protection pad illustrated in FIG. 1;

FIG. 5 is a side cross-sectional view along line A-A of the urinary incontinence protection pad illustrated in FIG. 2; and

FIG. 6 is a schematic cross-sectional view of a urinary incontinence protection pad according to another embodiment of the present invention.

### DETAILED DESCRIPTION OF THE INVENTION

Reference will now be made in detail to exemplary embodiments of the present disclosure, examples of which are illustrated in the accompanying drawings. Wherever possible, like reference numerals designate like elements throughout the drawings and description. The described embodiments are illustrative and explanatory and are not intended to limit the scope of the invention.

As used herein, the singular forms “a,” “an,” and “the” include plural referents unless the context clearly indicates otherwise. Terms such as “comprising,” “including,” and “having” are intended to be inclusive rather than exclusive.

As used herein, directional or relational terms such as “proximal,” “distal,” “inner,” “outer,” “first,” and “second” are used for convenience of description and not by way of limitation. During use, a proximal portion may refer to a portion closer to the user's body, and a distal portion may refer to a portion farther from the user's body.

As used herein, the term “male user” includes adult men, boys, and other users having male genital anatomy for which the disclosed device is suitable.

As used herein, the term “elongated tubular sleeve” refers to a generally tube-shaped body having an opening at one end and defining a space configured to receive urine released from a penis. The elongated tubular sleeve may be flexible and wearable.

As used herein, the term “elastic ring” refers to an annular or ring-like elastic structure disposed around the opening of the elongated tubular sleeve and configured to elastically engage the penis to retain the device in place and help inhibit leakage at the opening.

As used herein, the term “absorbent layer” refers to a layer or portion configured to receive and absorb urine.

As used herein, the term “absorbent core” refers to a fluid-retaining portion configured to absorb and retain urine within the device.

As used herein, the term “leak-resistant barrier” refers to a layer or portion of the device configured to resist passage of urine relative to adjacent absorbent structure and thereby help retain absorbed urine within the device.

Reference will now be made in detail to the exemplary embodiments of the present general inventive concept, examples of which are illustrated in the accompanying drawings, wherein like reference numerals refer to the like elements throughout. The exemplary embodiments are described below in order to explain the present general inventive concept by referring to the figures.

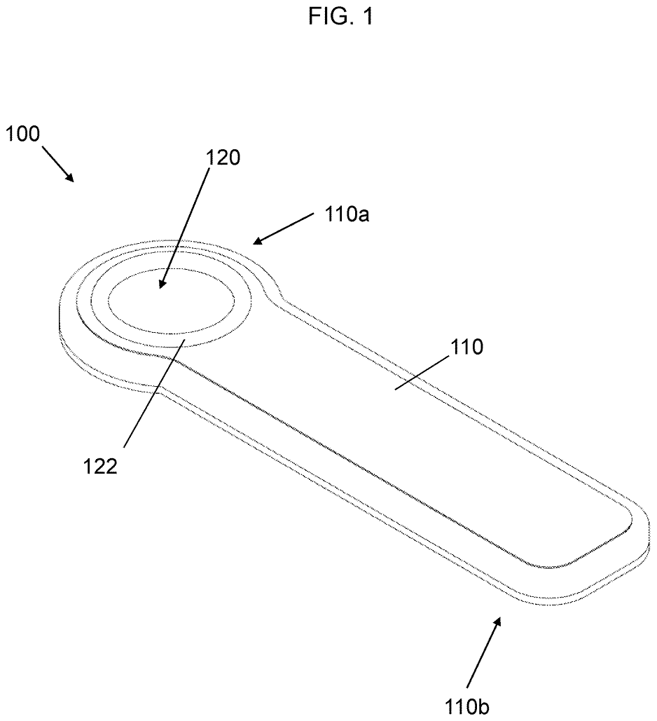

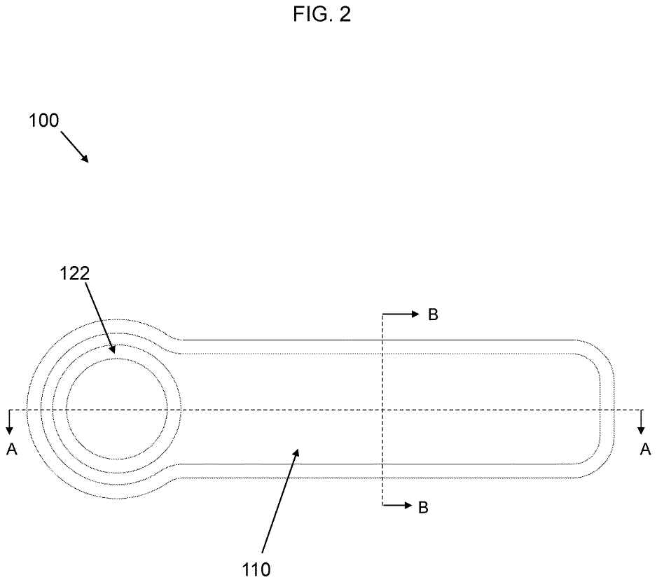

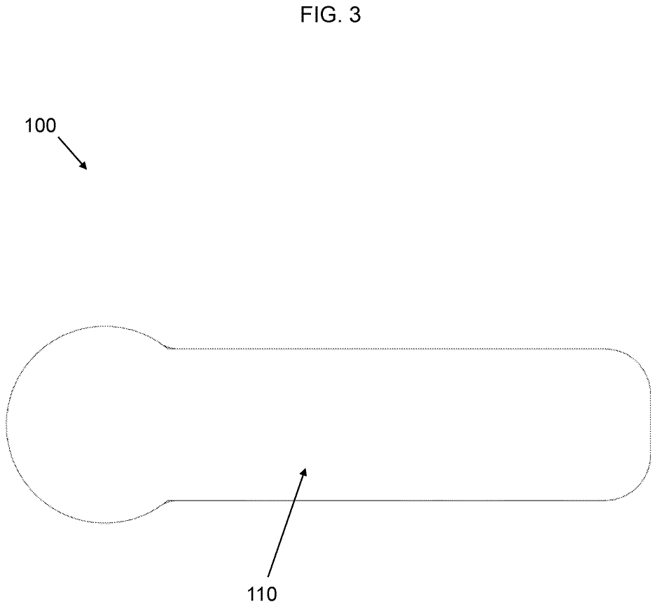

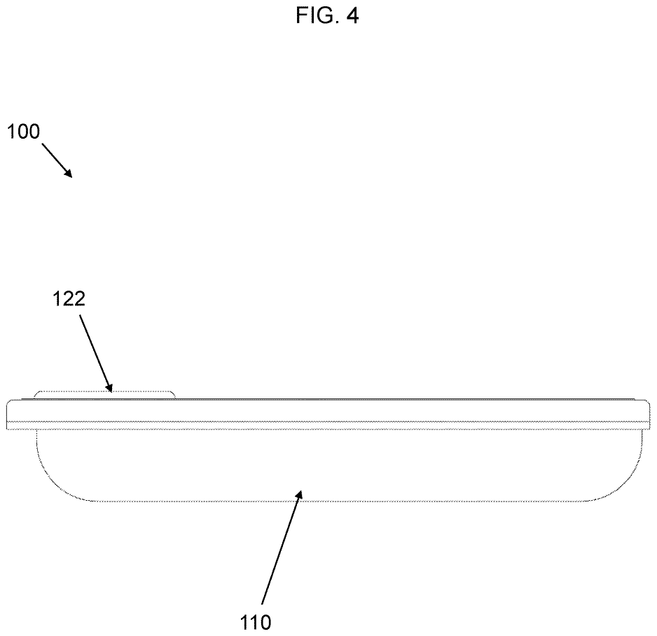

FIG. 1 is a front perspective view a urinary incontinence protection pad 100 according to an embodiment of the present inventive concept, FIG. 2 is a top view the urinary incontinence protection pad 100 illustrated in FIG. 1, and FIG. 3 is a bottom view of the urinary incontinence protection pad 100 illustrated in FIG. 1. FIG. 4 is a side view of the urinary incontinence protection pad 100 illustrated in FIG. 1.

Referring initially to FIGS. 1-4, a male urinary incontinence protection pad 100 according to an embodiment of the present disclosure is illustrated. The protection pad 100 is configured to be worn on a penis and to absorb urine released from the penis so as to help protect clothing, bedding, seating surfaces, and other surrounding materials.

The protection pad 100 may be particularly useful for nighttime urinary incontinence, bedwetting protection, long trips, travel, extended periods without convenient restroom access, and other situations in which a male user desires discreet absorbent protection.

The protection pad 100 may also be useful for male users experiencing weak bladder control, impaired bladder function, urinary leakage, urinary urgency, urinary incontinence, accidental urine release, or similar conditions or circumstances. However, the disclosed protection pad 100 is not limited to any particular diagnosis, severity, age group, or use environment.

In general, the protection pad 100 is configured as a wearable absorbent protective article that may be placed on the penis, worn during normal activity, and removed after a leakage event or urination event. The protection pad 100 is intended to provide protection by absorbing urine and helping reduce leakage outward from the protection pad 100.

In the illustrated embodiment, the protection pad 100 includes an elongated tubular sleeve 110. The elongated tubular sleeve 110 has a first or proximal end 110a defining an opening 120. The opening 120 is sized and configured to receive at least a portion of the penis of the user.

An elastic ring 122 is disposed around the opening 120. The elastic ring 122 is configured to elastically engage the penis when the penis is inserted through the opening 120. By engaging the penis, the elastic ring 122 retains the elongated tubular sleeve 110 on the penis during wear and assists in inhibiting leakage of urine from the opening 120.

In some embodiments, the elastic ring 122 continuously surrounds the opening 120. In some embodiments, the elastic ring 122 provides a circumferential securement region at the proximal end of the protection pad 100. In some embodiments, the elastic ring 122 forms a generally circular or substantially circular rim around the opening 120. However, the disclosed protection pad 100 is not limited thereto.

The elongated tubular sleeve 110 comprises absorbent material and is configured to absorb urine released from the penis during use. The elongated tubular sleeve 110 may extend longitudinally away from the opening 120 and may define an absorbent receiving space for urine.

In some embodiments, the elongated tubular sleeve 110 is sufficiently flexible to conform during wear and to permit discreet use under sleepwear, clothing, or other garments.

In some embodiments, the elongated tubular sleeve 110 is configured to be rolled up after use for easier and more sanitary disposal.

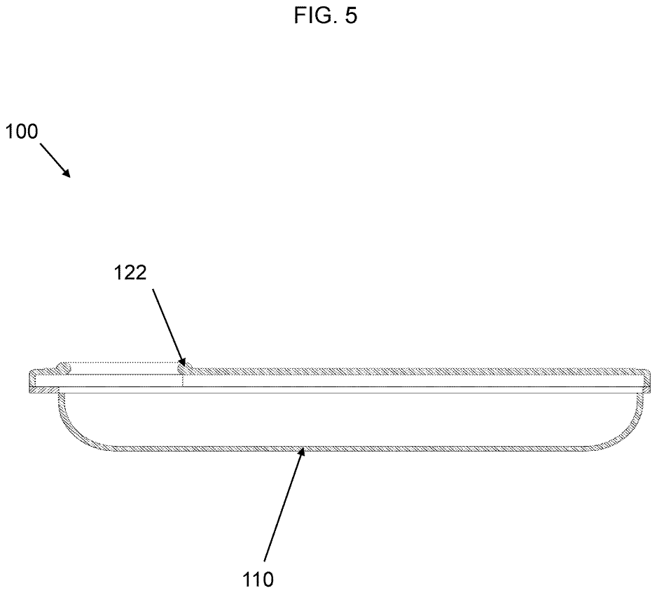

FIG. 5 is a side cross-sectional view along line A-A of the urinary incontinence protection pad 100 illustrated in FIG. 2.

Referring now to FIG. 5, the elongated tubular sleeve 110 may include one or more absorbent portions 112 configured to receive and retain urine.

In some embodiments, the elongated tubular sleeve 110 includes an absorbent layer 112. The absorbent layer 112 may be arranged within the elongated tubular sleeve 110 so as to receive urine entering the protection pad 100 and absorb the urine.

In some embodiments, the elongated tubular sleeve 110 includes an absorbent core 112. The absorbent core 112 may provide increased urine retention capacity and may extend along at least a portion of the elongated tubular sleeve 110.

In some embodiments, the protection pad 100 includes an absorbent layer 116 without a separately identified absorbent core. In some embodiments, the protection pad 100 includes an absorbent core 112 without a separately identified absorbent layer. In some embodiments, the protection pad 100 includes both an absorbent layer 116 and an absorbent core 112.

In embodiments having both an absorbent layer and an absorbent core, the absorbent layer may initially receive urine and the absorbent core may retain at least a portion of the urine. However, the present disclosure is not limited to any particular internal arrangement so long as the elongated tubular sleeve is configured to absorb urine released from the penis.

In some embodiments, the elongated tubular sleeve 110 further includes one or more leak-resistant barriers configured to help contain urine within the protection pad 100 after absorption.

As shown for example in FIG. 3, the elongated tubular sleeve 110 may include an inner leak-resistant barrier 114a and an outer leak-resistant barrier 114b.

In some embodiments, the absorbent core 112 is disposed between the inner leak-resistant barrier 114a and the outer leak-resistant barrier 114b. This arrangement may help retain urine absorbed by the absorbent core 112 and reduce the likelihood that urine will pass outwardly through the elongated tubular sleeve 110.

In some embodiments, the inner leak-resistant barrier 114a is positioned toward an interior region of the protection pad 100 and the outer leak-resistant barrier 114b is positioned toward an exterior region of the protection pad 100.

In some embodiments, the inner leak-resistant barrier 114a and the outer leak-resistant barrier 114b cooperate with the absorbent layer 116, the absorbent core 112, or both, to resist leakage from the protection pad 100 during use.

In some embodiments, the elongated tubular sleeve includes one leak-resistant barrier. In some embodiments, the elongated tubular sleeve includes multiple leak-resistant barriers. In some embodiments, the elongated tubular sleeve includes both an inner leak-resistant barrier and an outer leak-resistant barrier together with an absorbent core disposed therebetween.

In some embodiments, the absorbent structure of the protection pad 100 includes one or more absorbent-enhancing materials.

By way of example and without limitation, such absorbent-enhancing materials may include absorbent chemicals, gels, superabsorbent polymers, or combinations thereof.

In some embodiments, these materials may be disposed within an absorbent layer, within an absorbent core, or within another absorbent portion of the elongated tubular sleeve.

Inclusion of absorbent chemicals, gels, superabsorbent polymers, or combinations thereof may increase urine uptake, increase retention, or both.

The elastic ring 122 disposed around the opening 120 serves as a primary securement feature in certain embodiments. When the penis is inserted through the opening 120, the elastic ring 122 engages the penis to help hold the protection pad 100 in place.

The elastic ring 122 also helps reduce leakage at the proximal end of the protection pad 100 by maintaining engagement around the penis adjacent the opening 120.

Referring to FIG. 5, another embodiment of a male urinary incontinence protection pad 200 is illustrated. The protection pad 200 may include features the same as or similar to those described above with respect to protection pad 100, except as otherwise noted.

In the embodiment of FIG. 5, the protection pad 200 includes an absorbent layer 216 and an elastic band portion 218.

The elastic band portion 118 may be disposed along edges of the elongated tubular sleeve 110 and may further secure the protection pad 100 on the penis during use.

In some embodiments, the elastic band portion 118 cooperates with the elastic ring surrounding the opening to help maintain the elongated tubular sleeve in position during movement, sleep, sitting, travel, or other activity.

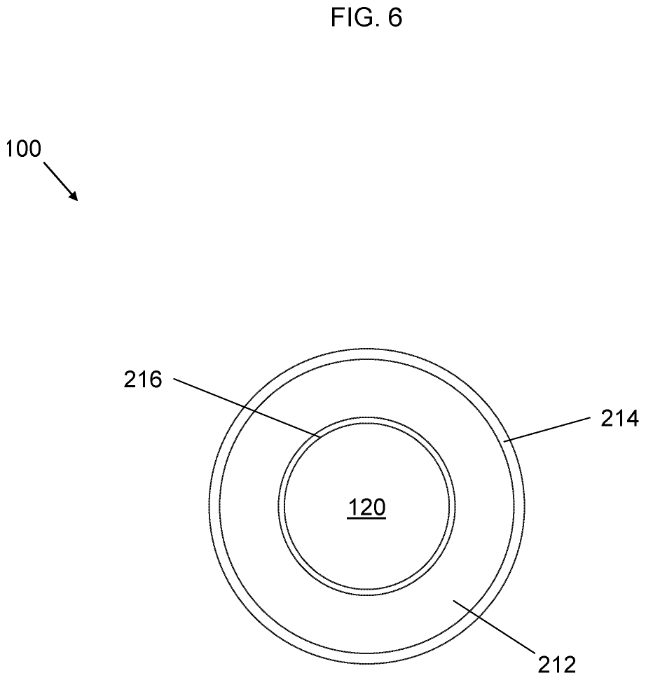

FIG. 6 is a schematic cross-sectional view of an urinary incontinence protection pad 200 according to another embodiment of the present invention.

Referring to FIG. 6, a further embodiment of a male urinary incontinence protection pad 200 is illustrated. The protection pad 200 may include features the same as or similar to those described above with respect to protection pad 100, except as otherwise noted.

In the embodiment of FIG. 6, the protection pad 200 includes an absorbent layer 212, an elastic band portion 220, and absorbent chemicals and/or gel associated within the absorbent layer 212.

The protection pad 200 further includes an inner leak-resistant barrier 214 and an outer leak-resistant barrier 216 that cooperate with the absorbent material. The inner leak-resistant barrier 214 may allow fluids, including urine to only flow in one-direction, toward the absorbent layer 212.

The embodiment of FIG. 6 illustrates that the protection pad may include multiple absorbent and securement-related features in combination, including an elongated tubular sleeve, an opening surrounded by an elastic ring, an absorbent layer, an elastic band portion, and absorbent chemicals and/or gel materials.

In use, a male user may remove the protection pad from packaging and position the penis through the opening at the proximal end of the elongated tubular sleeve.

As the penis passes through the opening, the elastic ring surrounding the opening elastically engages the penis and retains the protection pad on the penis.

Once in place, the protection pad may be worn during sleep, rest, travel, long trips, driving, sitting, medical care, or other normal activities.

When urine leakage or involuntary urination occurs, urine released from the penis enters the elongated tubular sleeve and is absorbed by the absorbent structure of the protection pad.

In embodiments having an absorbent layer, the absorbent layer absorbs the urine. In embodiments having an absorbent core, the absorbent core retains the urine. In embodiments having both, the absorbent layer and absorbent core may cooperate to absorb and retain the urine.

In embodiments having an inner leak-resistant barrier and an outer leak-resistant barrier, the barriers cooperate with the absorbent structure to help contain the urine within the protection pad.

In embodiments having an elastic band portion, the elastic band portion may provide further securement during use.

After use, the protection pad may be removed from the penis. In some embodiments, the protection pad may be rolled up and discarded in a sanitary and discreet manner.

The present disclosure contemplates numerous embodiments and combinations consistent with the structures shown and described herein.

In some embodiments, the protection pad comprises an elongated tubular sleeve, an opening at a proximal end, and an elastic ring surrounding the opening.

In some embodiments, the protection pad comprises an elongated tubular sleeve, an opening at a proximal end, an elastic ring surrounding the opening, and an absorbent layer.

In some embodiments, the protection pad comprises an elongated tubular sleeve, an opening at a proximal end, an elastic ring surrounding the opening, and an absorbent core.

In some embodiments, the protection pad comprises an elongated tubular sleeve, an opening at a proximal end, an elastic ring surrounding the opening, and both an absorbent layer and an absorbent core.

In some embodiments, the protection pad comprises an elongated tubular sleeve, an opening at a proximal end, an elastic ring surrounding the opening, an absorbent core, an inner leak-resistant barrier, and an outer leak-resistant barrier.

In some embodiments, the protection pad comprises an elongated tubular sleeve, an opening at a proximal end, an elastic ring surrounding the opening, an absorbent layer, and an elastic band portion.

In some embodiments, the protection pad comprises an elongated tubular sleeve, an opening at a proximal end, an elastic ring surrounding the opening, an absorbent layer, and absorbent chemicals and/or gel materials.

In some embodiments, the protection pad comprises an elongated tubular sleeve, an opening at a proximal end, an elastic ring surrounding the opening, an absorbent layer, an elastic band portion, and absorbent chemicals and/or gel materials.

In some embodiments, the protection pad comprises an elongated tubular sleeve, an opening at a proximal end, an elastic ring surrounding the opening, an absorbent core, an absorbent layer, and one or more leak-resistant barriers.

In some embodiments, the protection pad comprises an elongated tubular sleeve, an opening at a proximal end, an elastic ring surrounding the opening, an absorbent core, an inner leak-resistant barrier, an outer leak-resistant barrier, and one or more of an absorbent chemical, gel, superabsorbent polymer, or elastic band portion.

Features described with respect to one embodiment may be used together with features described with respect to another embodiment unless incompatible. Accordingly, the illustrated embodiments should not be viewed as mutually exclusive.

The disclosed protection pad may be used in a variety of settings in which urinary incontinence protection is desired.

By way of example, the protection pad may be used during nighttime sleep to address bedwetting or nighttime urinary leakage.

By way of further example, the protection pad may be used during long trips, including automobile travel or other transportation settings in which restroom access may be delayed.

By way of further example, the protection pad may be useful for drivers, passengers, patients, campers, athletes, military personnel, travelers, or other users who desire temporary and discreet urinary protection.

By way of further example, the protection pad may be used in home care, hospital care, assisted living, or similar care settings to help protect clothing, bedding, and nearby surfaces.

These examples are illustrative only and do not limit the scope of the disclosed invention.

The present disclosure also encompasses methods of providing urinary incontinence protection for a male user.

In one method embodiment, a penis of a male user is placed through an opening of a urinary incontinence protection pad.

An elastic ring surrounding the opening engages the penis and retains the protection pad on the penis.

Urine released from the penis is received within an elongated tubular sleeve of the protection pad.

The urine is absorbed by absorbent material of the elongated tubular sleeve, including for example an absorbent layer, an absorbent core, or both.

In some method embodiments, the protection pad further includes an inner leak-resistant barrier and an outer leak-resistant barrier that cooperate with the absorbent material.

In some method embodiments, the protection pad further includes an elastic band portion that further secures the protection pad on the penis.

In some method embodiments, the absorbent material includes absorbent chemicals, gel materials, superabsorbent polymers, or combinations thereof.

In some method embodiments, the protection pad is worn during sleep.

In some method embodiments, the protection pad is worn during a long trip or extended travel period.

In some method embodiments, the protection pad is removed after use and rolled up for disposal.

For clarity and claim support, the elongated tubular sleeve described herein may also be understood as a sleeve body defining a generally tubular absorbent structure configured to be worn on the penis and absorb urine released therefrom.

The opening at the first end of the elongated tubular sleeve may also be referred to as a proximal opening.

The elastic ring disposed around the opening may also be described as surrounding the proximal opening and being configured to secure the sleeve body to the penis and reduce leakage at the proximal opening.

The absorbent core may be carried by the sleeve body and positioned relative to the inner leak-resistant barrier and outer leak-resistant barrier such that urine absorbed by the absorbent core is retained within the protection pad.

The disclosure therefore supports apparatus claims, protection pad claims, and method claims using the foregoing terminology.

In some embodiments, the protection pad is configured as a disposable absorbent article intended for temporary use.

After use, the protection pad may be removed and rolled up to reduce visibility of the used article and facilitate sanitary disposal.

The portable and discreet nature of the protection pad may allow a user to carry the protection pad before use and dispose of the protection pad after use without drawing unnecessary attention.

The drawings illustrate exemplary configurations of the protection pad and should not be understood as limiting the invention to only the exact arrangements shown.

Features shown in one figure may be combined with features shown in another figure to form additional embodiments consistent with the present disclosure.

The scope of the invention is defined by the appended claims and their equivalents, and not by the foregoing examples alone.

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A male urinary incontinence protection pad, comprising:
an elongated tubular sleeve comprising absorbent material and having an opening at a first end, the elongated tubular sleeve being sized to receive at least a portion of a penis through the opening; and an elastic ring disposed around the opening and configured to elastically engage the penis to retain the elongated tubular sleeve on the penis and inhibit leakage of urine from the opening,
wherein the elongated tubular sleeve is configured to absorb urine released from the penis.

2. The male urinary incontinence protection pad of claim 1, wherein the elongated tubular sleeve comprises an absorbent layer.

3. The male urinary incontinence protection pad of claim 1, wherein the elongated tubular sleeve comprises an absorbent core.

4. The male urinary incontinence protection pad of claim 3, wherein the elongated tubular sleeve further comprises an inner leak-resistant barrier and an outer leak-resistant barrier.

5. The male urinary incontinence protection pad of claim 4, wherein the absorbent core is disposed between the inner leak-resistant barrier and the outer leak-resistant barrier.

6. The male urinary incontinence protection pad of claim 1, wherein the absorbent material comprises a gel, a superabsorbent polymer, an absorbent chemical, or a combination thereof.

7. The male urinary incontinence protection pad of claim 1, further comprising an elastic band portion disposed on the elongated tubular sleeve and configured to further secure the elongated tubular sleeve on the penis.

8. The male urinary incontinence protection pad of claim 1, wherein the elastic ring continuously surrounds the opening.

9. The male urinary incontinence protection pad of claim 1, wherein the device is configured for nighttime urinary incontinence protection.

10. The male urinary incontinence protection pad of claim 1, wherein the device is configured to help protect clothing, bedding, or seating surfaces from urine leakage.

11. A urinary incontinence protection device for a male user, comprising:
a sleeve body defining an elongated tubular sleeve having a proximal opening sized to receive at least a portion of a penis;
an absorbent core carried by the sleeve body;
an inner leak-resistant barrier and an outer leak-resistant barrier positioned on opposite sides of the absorbent core; and
an elastic ring surrounding the proximal opening and configured to elastically engage the penis to secure the sleeve body to the penis and reduce leakage at the proximal opening.

12. The urinary incontinence protection device of claim 11, wherein the sleeve body further comprises an absorbent layer.

13. The urinary incontinence protection device of claim 11, wherein the absorbent core includes or is associated with a gel, a superabsorbent polymer, an absorbent chemical, or a combination thereof.

14. The urinary incontinence protection device of claim 11, further comprising an elastic band portion disposed on the sleeve body and configured to further secure the sleeve body on the penis.

15. The urinary incontinence protection device of claim 11, wherein the device is configured to be removed after use and rolled up for disposal.

16. The urinary incontinence protection device of claim 11, wherein the device is configured to help protect clothing, bedding, or seating surfaces from urine leakage.

17. The urinary incontinence protection device of claim 11, wherein the device is configured for nighttime use or long-trip use.

18. A method of providing urinary incontinence protection for a male user, the method comprising:
placing a penis of the male user through an opening of a urinary incontinence protection device such that the penis is engaged by an elastic ring surrounding the opening;
retaining the urinary incontinence protection device on the penis with the elastic ring; and
absorbing urine released from the penis within an elongated tubular sleeve of the urinary incontinence protection device.

19. The method of claim 18, wherein the elongated tubular sleeve comprises an absorbent layer, an absorbent core, or both.

20. The method of claim 18, wherein the urinary incontinence protection device further comprises an inner leak-resistant barrier and an outer leak-resistant barrier.
